(19) United States
(12) Patent Application Publication
(10) Pub. No.: US 2014/0180636 A1
(43) Pub. Date: Jun. 26, 2014

(54) METHODS AND SYSTEMS FOR ANALYZING DETECTION ENHANCEMENT OF MICROCANTILEVERS WITH LONG-SLIT BASED SENSORS

(71) Applicant: Kambix Innovations, LLC, Albuquerque, NM (US)

(72) Inventors: Kambiz Vafai, Mission Viejo, CA (US); Abdul Rahim A. Khaled, Jeddah (SA)

(73) Assignee: Kambix Innovations, LLC, Albuquerque, NM (US)

(21) Appl. No.: 14/042,837

(22) Filed: Oct. 1, 2013

Related U.S. Application Data
(60) Provisional application No. 61/709,374, filed on Oct. 4, 2012.

(51) Int. Cl. G01N 33/543; G01N 19/00

(52) U.S. Cl. CPC G01N 33/54373; G01N 19/00
USPC 702/182

(57) ABSTRACT

Methods and systems for analyzing the detection enhancement of rectangular microcantilevers with long-slit microsensors. The deflection profile of the microcantilevers can he compared with that of typical rectangular microcantilevers under presence of dynamic disturbances. Various force-loading conditions are considered. The theory of linear elasticity for thin beams is used to obtain the deflection related quantities. The disturbance in these quantities can be obtained based on wave propagation and beam vibration theories.

### CROSS-REFERENCE TO PROVISIONAL PATENT APPLICATION

This patent application claims the benefit under 35 U.S.C. §119(e) of U.S. Provisional Application Ser. No. 61/709,374 entitled, “Analysis of Detection Enhancement Using Microcantilevers with Long-Slit Based Sensors,” which was filed on Oct. 4, 2012 and is incorporated herein by reference in its entirety.

### FIELD OF THE INVENTION

Embodiments are generally related to microsensors. Embodiments also relate to rectangular microcantilevers with long-slit utilized in microsensing applications. Embodiments are additionally related to systems and methods for analyzing detection enhancement of rectangular microcantilevers with long-slit in microsensing applications.

### BACKGROUND

The recent advances in nanoscience and nanotechnologies have revealed development of innovative and highly sensitive microsensors. These microsensors are now becoming pivotal tools in exploring many chemical and biological phenomena. An example of these sensors is the microcantilever. The deflection of the microcantilever was first used for atomic force microscopy. See Alkarnine, S., Barrett, R. C., and Quate, C. F., Improved atomic force microscope images using microcantilevers with sharp tips, Appl. Phys. Lett., 1990, 57, 316-318. Microcantilevers are now being used universally to accurately assay the unknown species present in a medium. See Arntz, Y., Seelig, J. D., Lang, H. P., Zhang, J., Hunziker, P., Ramseyer, J. P., Meyer, E., Hegner, M., and Gerber, C., Label-free protein assay based on a nanomechanical cantilever array, Nanotechnology, 2003, 14, 86-90; Sun, C. R., Kaur, J., Gandhi, S., and Shekhawat, G. S., Label-free ultra-sensitive detection of atrazine based on nanomechanics, Nanotechnology, 2008, 19, 235502-235600: and Calleja, M., Nordstrom, M., Alvarez, M., Tamayo, J., Lechuga, L. M., and Boisen, A., Highly sensitive polymer-based cantilever-sensors for DNA detection, Ultramicroscopy, 2005, 105, 215-222.

Moreover, they can be used to monitor presence of specific diseases inside a human body at early stages. See Wu, G., Ji, H., Hansen, K., Thundat, T., Datar, R., Cote, R., Hagan, M. F., Chakraborty, A. K., and Majurndar, A., Origin of nanomechanical cantilever motion generated from biomolecular interactions, Proc. Natl. Acad. Sci. USA, 2001, 98, 1560-1564,

The sensing feature of the microcantilevers is generally based on measuring the deflection caused by the adhesion of specific species called either analyte or target on the receptor coating layer of the microcantilever. See Boisen, A., Thaysen, Jensenius, H., and Hansen, O., Environmental sensors based on miromachined cantilevers with integrated read-out, Ultramicroscopy, 2000, 82, 11-16; and Zuo, G., Li, X., Zhang, Z., Yang, T., Wang, Y., Cheng, Z,, and Feng, S., Dual-SAM functionalization on integrated cantilevers for specific trace-explosive sensing and non-specific adsorption suppression, Nanotechnology, 2007, 18, doi:10.1088/0957-4484/18/25/255501.

The analyte-receptor adhesion produces compression/tension surface stress, thus bending of microcantiever occurs which causes the microcantilever to deflect. See Fritz, J., Mier, M. K., Lang, H. P,, Rothuizen, H., Vettiger, R, Meyer, E., Guntherodt, H.-J., Gerber, C., and Gimzewski, J. K., Translating biomolecular recognition into nanomechanics, Science, 2000, 288, 316-318.

The adherence-induced deflections can be measured using optical techniques or using electric signals with piezoresistive microcantilevers. See Khaled, A.-R. A., Vafai, K., Yang, M., Zhang, X., and Ozkan, C. S., Analysis, control and augmentation of microcantilever deflections in bio-sensing systems, Sens. Actuat. B, 2003, 94, 103-115; and Boisen, A., Thaysen, J., Jensenius, H., and Hansen, a, Environmental sensors based on micromachined cantilevers with integrated read-out, Ultramicroscopy, 2000, 82, 11-16. Also see Lee, H. J, Chang, Y. S., Lee, Y. P., Jeong, K.-H., and Kim, H. -Y., Deflection of microcantilever by growing vapor bubble, Sens. Actuat. A, 2007, 136, 717-722; and Jean, S., Jung, N., and Thundat, T., Nanomechanics of self-assembled monolayer on microcantilever sensors measured by a multiple-point deflection technique, Sens. Actuat. B, 2007, 122, 365-368.

These deflections are frequently ranged from few tens to few hundreds of nanometers. Accordingly, increasing the sensitivity of the microcantilever detection becomes a major challenge when it is used to detect or monitor low concentrations of analyte. See Fritz, J., Bailer, M. K., Lang, H. P., Rothuizen, H., Vettiger, P., Meyer, E., Guntherodt, H.-J,, Gerber, C., and Gimzewski, J. K., Translating biomolecular recognition into nanomechanics, Science, 2000, 288, 316-318;, Lee, H. J, Chang, Y. S., Lee, Y. P., Jeong, K.-H., and Kim, H.-Y., Deflection of microcantilever by growing vapor bubble, Sens. Actual. A, 2007, 136, 717-722; and Jeon, S., Jung, N., and Thundat, T., Nanomechanics of self-assembled monolayer on microcantilever sensors measured by a multiple-point deflection technique, Sens. Actuat. B, 2007, 122, 365-368.

The detection capability of the microcantilever is influenced by the disturbance level in the adjacent medium. Fritz at al. indicated that the deflection of the microcantilever due to external excitations could reach 5-10 times the microcantilever deflection due to analyte-receptor adhesion. The basic constituents of these excitations are the flow disturbances, acoustic wave disturbances, and variations in the microcantilever thermal conditions prior and after injection of the analyte solution. The flow disturbances and acoustic wave disturbances are usually called dynamic disturbances. Further developments in microcantilever technology were carried so that the deflection signal due to the microsensing effect can be magnified. Therefore, the microsensing deflection signal can be easily distinguished from the disturbance (noise) in deflection signal. See Yang, M., Zhang, X., Vafai, K., and Ozkan, C. S., High sensitivity piezoresistive cantilever design and optimization for an analyte-receptor binding, J. Micromech. Microeng., 2003, 13, 864-872; Vafai, K., Ozkan, C., Haddon, R., Khaled, A.-R. A., and Yang, M., Microcantilevers for Biological and Chemical Assays and Methods of Making and Using Thereof, U.S. Pat. No. 7,288,404, 30 Oct. 2007; Zhu, Q., Shih, W. Y., and Shih, W.-Y., Enhanced detection resonance frequency shift of a piezoelectric microcantilever sensor by a DC bias electric field in humidity detection, Sens. Actual. B, 2008, 138, 1-4; Yen, Y.-K., Huang C.-Y,, Chen, C.-H., Hung, C.-M., Wu, K.-C., Lee, C.-K., Chang, J.-S., Lin, S., and Huang L.-S., A novel, eclectically protein-manipulated microcantilever biosensor for enhancement of capture antibody immobilization, Sens. Actual. B, 2009, 141, 498-505; and Ansari, M. Z., and Cho, C., Deflection, Frequency, and Stress Characteristics of Rectangular, Triangular, and Step Profile Microcantilevers for Biosensors. Sensors, 2009, 9, 6046-6057.

Consequently, Khaled et al. emphasized the necessity to design special microcantilever assemblies for this purpose. Many of these assemblies were analyzed and validated. See Vafai, K,, Ozkan, C., Haddon, R., Khaled, A.-R. A., and Yang, M., Microcantilevers for Biological and Chemical Assays and Methods of Making and Using Thereof, U.S. Pat. No. 7,288,404, 30 Oct. 2007; and Vafai, K., and Khaled, A.-R. A., Innovative biosensors for chemical and biological assays, U.S, Pat. No. 7,695,951, 13 Apr. 2010.

Moreover, additional innovative methods for enlarging the deflection signal due to microsensing effect were discussed. Some of these methods are based on controlling both the geometry of the fluidic cell incubating the microcantilevers and their geometrical distribution. See Khaled, A.-R. A., and Vafai, K., Optimization modelling of analyte adhesion over an inclined microcantilever-based biosensor, J. Micromech. Microeng., 2004, 14, 1220-1229; Khanafer, K., Khaled, A.-R. A., and Vafai, K., Spatial optimization of an array of aligned microcantilever based sensors, J. Micromech. Microeng., 2004, 14, 1328-1336; Khanafer, K., and Vafai, K., Geometrical and flow configurations for enhanced microcantilever detection within a fluidic cell, Int, J. Heat Mass Transf., 2005, 48, 2886-2895; and Vafai, K., and Khaled, A.-R. A., Methods and devices comprising flexible seals, flexible microchannels, or both for modulating or controlling flow and heat, U.S. Pat. No. 7,770,809, 10 Aug. 2010.

A remarkable microcantilever assembly among the assemblies in the work of Khaled at al., Analysis, control and augmentation of microcantilever deflections in bio-sensing systems, Sens. Actuat, B, 2003, 94, 103-115 is the rectangular microcantilever with long-slit. The adherence-induced detection of this type of microcantilevers is almost unaffected by the dynamic disturbances. This type of microcantilever assemblies is made of rectangular microcantilever with the receptor coating being placed on one half of the upper surface of the microcantilever and along the opposite half of the lower surface of the microcantilever. Furthermore, this microcantilever has a long slit along the interface between the receptor coating and the remaining surface portion that is free from receptor. The long-slit allows the separated sides of the microcantilever to have deflections in opposite directions upon analyte adhesion with the receptors. These deflections are able to produce slit opening length (normal to deflection axis) that is much larger than deflections of typical rectangular microcantilevers. The slit opening length which can be correlated to analyte concentration is affected less by dynamic disturbances. This is because both surfaces are subjected to almost similar flow drags or similar acoustic waves. No additional works have been conducted to demonstrate these aspects.

Therefore, a need exists for an improved system and method for analyzing deflections of rectangular microcantilever with long-slit.

### BRIEF SUMMARY

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for microsensors.

It is another aspect of the disclosed embodiments to provide for detection enhancement by utilizing the rectangular microcantilevers with long slit for microsensing applications.

It is a further aspect of the present invention to provide for a method and system for analyzing detection enhancement by utilizing the rectangular microcantilevers with long slit for microsensing applications by comparing with typical rectangular cantilevers under the presence of dynamic disturbances.

The aforementioned aspects and other objectives and advantages can now be achieved as described herein. A method and system for analyzing system and method for analyzing detection enhancement of rectangular microcantilevers with long slit in microsensing applications is disclosed. The deflection profile of the microcantilevers is compared with that of typical rectangular microcantilevers under presence of dynamic disturbances. Various force-loading conditions are considered. The theory of linear elasticity for thin beams is used to obtain the deflection related quantities. The disturbance in these quantities is obtained based on wave propagation and beam vibration theories.

It is found that detections of rectangular microcantilevers with long-slit based on maximum slit opening length can be more than 100 times the deflections of the typical rectangular microcantilevers. Moreover, the disturbance (noise effect) in the detection quantities of the microcantilever with long-slit is found to be always smaller than that of typical microcantilevers regardless of the wave length, force amplitude, and the frequency of the dynamic disturbance. Eventually, the detection quantities of the microcantilever with long-slit are found to be almost unaffected by dynamic disturbances as long as the wave lengths of these disturbances are larger than 3.5 times the microcantilever width.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### BRIEF DESCRIPTION OF THE FIGURES

The accompanying figures, in which like reference numerals refer to identical or functionally-similar elements throughout the separate views and which are incorporated in and form a part of the specification, further illustrate the disclosed embodiments and, together with the detailed description of the invention, serve to explain the principles of the disclosed embodiments.

FIGS. 1A and 1B illustrate top and side views of a typical rectangular microcantilever in a corresponding coordinate system respectively, in accordance with the disclosed embodiments;

FIGS. 2A and 2B illustrate top and side views of a typical rectangular microcantilever with long-slit in a corresponding coordinate system respectively, in accordance with the disclosed embodiments;

FIG. 2C illustrates a deflection profile of rectangular microcantilever with long-slit with major deflection quantities, in accordance with the disclosed embodiments;

FIG. 3 illustrates a deflection profile of rectangular microcantilever with long-slit with Lₒ=425 μm, L=415 μm, W=60 μm, d=0.4 μm, c=5 μm, E=0.1124N·μm⁻², v=0.28, and F=2×10⁻⁹N, in accordance with the disclosed embodiments;

FIG. 4 illustrates a graph showing effects of maximum rectangular microcantilever with long-slit side beams dimensionless deflection {(z_{F})ₘₐₓ|_{LB}/d} and the slit profile dimensionless length (L/d) on the first detection enhancement indicator due to concentrated force loading (γ_{1,F}), in accordance with the disclosed embodiments;

FIG. 5 illustrates a graph showing effects of maximum rectangular microcantilever with long-slit side beams dimensionless deflection {(z_{Δσ})ₘₐₓ|_{LB}/d}, the slit profile dimensionless length (L/d), and power law index (n) on the first detection enhancement indicator due to prescribed surface stress loading (γ_{1,Δcr}), in accordance with the disclosed embodiments;

FIG. 6 illustrates a graph showing effects of the dimensionless frequency of dynamic disturbance (ω/ω₀) and the first dimensionless dynamic disturbance force amplitude {P₀ω₀²/F} on the dearness indicator of the deflection signal due to concentrated force loading (X_{F}), in accordance with the disclosed embodiments;

FIG. 7 illustrates a graph showing effects of the dimensionless frequency of dynamic disturbance (ω/ω₀), the second dimensionless dynamic disturbance force amplitude. {Pₒωₒ²(L/d)/[ΔσₒW(1−v]}, and power law index (n) on the dearness indicator of the deflection signal due to prescribed surface stress loading (X_{Δσ}), in accordance with the disclosed embodiments;

FIG. 8 illustrates a graph showing effects of the dimensionless dynamic disturbance wave length (λ/W) and the first dimensionless dynamic disturbance force amplitude {Pₒωₒ²/F} on the second detection enhancement indicator of the rectangular microcantilever with long-slit due to concentrated force loading (γ_{2,F}), in accordance with the disclosed embodiments;

FIG. 9 illustrates a graph showing effects of the dimensionless dynamic disturbance wave length (λ/W) and the second dimensionless dynamic disturbance force amplitude {Pₒωₒ²(L/d)/[ΔσₒW(1−v)]} on the second detection enhancement indicator of the rectangular microcantilever with long-slit due to prescribed surface stress loading (γ_{2,Δσ}), in accordance with the disclosed embodiments;

FIG. 10 illustrates a graph showing effects of the dimensionless frequency of dynamic disturbance (ω/ω₀) and the first dimensionless dynamic disturbance force amplitude {Pₒωₒ²/F} on the second detection enhancement indicator of the rectangular microcantilever with long-slit due to concentrated force loading (γ_{2,F}), in accordance with the disclosed embodiments; and

FIG. 11 illustrates a graph showing effects of the dimensionless frequency of dynamic disturbance (ω/ω₀), the power law index (n), and the second dimensionless dynamic disturbance force amplitude {Pₒωₒ²(L/d)/[ΔσₒW(1−v)]} on the second detection enhancement indicator of the rectangular microcantilever with long-slit due to prescribed surface stress loading (γ_{2,Δσ}), in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The following Table 1 provides the various symbols and meanings used in this section:

TABLE 1

| | |
|---|---|
| c | clearance length for rectangular microcantilever with long-slit (m) |
| d | microcantilever thickness (μm) |
| E | Elastic modulus (N μm⁻²) |
| F | concentrated force (N) |
| l | Area moment of inertia (μm⁴) |
| k | stiffness (N/μm) |
| L | typical rectangular microcantilever or slit length (μm) |
| Lₒ | length of rectangular microcatilever with long-slit (m) |
| M | moment (N μm) |
| m | mass (kg) |
| n | surface stress model index |
| Pₒ | dynamic disturbance force per frequency of disturbance (N.s) |
| t | Time variable (s) |
| W | total microcantilever width (μm) |
| x | axis of the extension dimension (μm) |
| Y | effective elastic modulus (N μm⁻²) |
| Z | first deflection indicator |
| z | deflection (μm) |
| z_{d} | Amplitude of disturbance in deflection (μm) |

Greek Symbols

| | |
|---|---|
| χ | detection clearness indicator |
| δ | slit thickness (μm) |
| γ₁ | the first detection enhancement indicator |
| γ₂ | the second detection enhancement indicator |
| λ | wave length of the dynamic disturbance (μm) |
| v | Poisson's ratio |
| σ | surface stress |
| ω | dynamic disturbance frequency (s⁻¹) |
| ωₒ | first natural frequency of typical rectangular microcantilever (s⁻¹) |
| ωₛ | first natural frequency of rectangular microcantilever with long-slit |
| | (s⁻¹) |

Subscripts

| | |
|---|---|
| d | disturbance |
| F | concentrated force condition |
| Δσ | prescribed surface stress condition |
| eff | effective value |

Abbreviations

| | |
|---|---|
| LB | Left beam of the rectangular microcantilever with long-slit |
| RB | Left beam of the rectangular microcantilever with long-slit |

1. Theoretical Analysis
1.1. The Typical Rectangular Microcantilever

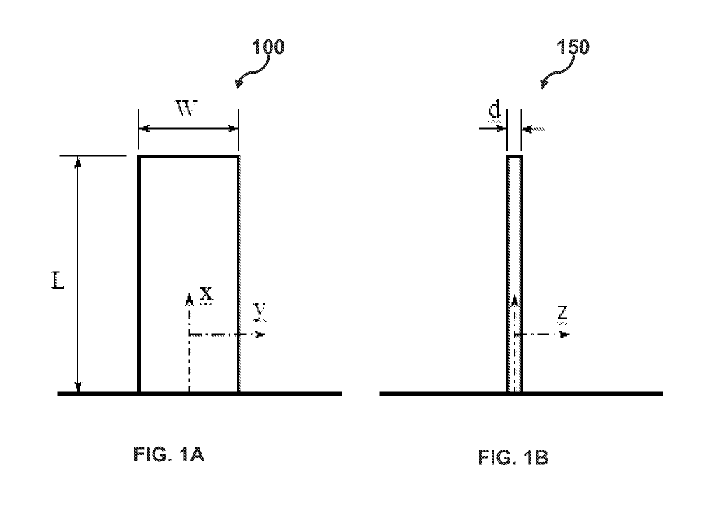

FIGS. 1A and 1B illustrate top and side views 100 and 150 of a typical rectangular microcantilever in a corresponding coordinate system, respectively. The properties of this microcantilever are given by the extension length L, width W, thickness d, Young's modulus E, and Poisson's ratio v.

### 1.1.1. Deflections of the Typical Rectangular Microcantilever

When the length of the microcantilever is much larger than its width, Hooks law for small deflections can be used to relate the microcantilever deflection at a given cross-section to the effective elastic modulus Y of the microcantilever and the bending moment M acting on that section, see Khaled, A.-R. A., and Vafai, K., Analysis of Deflection Enhancement Using Epsilon Assembly Microcantilevers Based Sensors, Sensors, 2011, 11, 9260-9274.

It is given by:

$$\frac{{d}^{2} z}{d {x}^{2}} = \frac{M}{YI} \qquad Eq . ( 1 )$$

where I is the area moment of inertia of the microcantilever cross-section about its neutral axis. It is given by:

$$I = \frac{1}{12} {Wd}^{3} \qquad Eq . ( 2 )$$

The boundary conditions for Equation (1) are given by:

$$z ( x = 0 ) = \frac{d z}{d x} {\vert}_{x = 0} = 0 \qquad Eq . ( 3 a , b )$$

The magnitude of microcantilever stress at bottom surface (z=d/2) or upper surface (z=−d/2), associated with the bending moment M can be calculated from the following equation:

$$σ = \frac{M}{I} ( \frac{d}{2} ) \qquad Eq . ( 4 )$$

Concentrated force loading:

If a concentrated force in the direction of the z-axis is exerted on the microcantilever tip located at x=L, then the internal bending moment M at any cross-section is linearly increasing from the tip to the base x=0. The internal bending moment distribution is equal to:

$$M = FL ( 1 - \frac{x}{L} ) \qquad Eq . ( 5 )$$

For this case, the effective elastic modulus is the same as the elastic modulus (Y=E). The magnitude of maximum stress occurs at (x,z)=(0,±d/2). It is denoted by σ_{oF}. Using Equation (4), σ_{oF} can be shown to be equal to:

$${σ}_{oF} = \frac{6 FL}{{Wd}^{2}} \qquad Eq . ( 6 )$$

The solution of Equation (1), denoted by z_{F}(x), can be expressed as:

$${z}_{F} ( x ) = ( \frac{6 {FL}^{3}}{{EWd}^{3}} ) [ {( \frac{x}{L} )}^{2} - \frac{1}{3} {( \frac{x}{L} )}^{3} ] \qquad Eq . ( 7 )$$

The maximum deflection (z_{F})ₘₐₓ which occurs at x=L can be expressed as:

$${( {z}_{F} )}_{ma x} = \frac{4 {FL}^{3}}{{EWd}^{3}} \qquad Eq . ( 8 )$$

Define the concentrated force deflection indicator Z_{F} as the ratio of the maximum microsensor deflection per maximum stress under constant concentrated force applied at the microcantilever tip. Using Equations (6) and (8), Z_{F} can be shown to be equal to:

$${Z}_{F} ≡ \frac{{( {z}_{F} )}_{ma x}}{{σ}_{oF}} = ( \frac{2}{3} ) \frac{{L}^{2}}{Ed} \qquad Eq . ( 9 )$$

Prescribed differential surface stress:

When one side of the microcantilever is coated with a thin film of receptor, the microcantilever will bend if the analyte molecules adhere on that layer. This adhesion causes a difference in the surface stresses across the microcantilever cross-section (Δσ). This results in an internal bending moment M at each cross-section. M is related to Δσ through the following equation:

$$M = \frac{Δ σ Wd}{2} \qquad Eq . ( 10 )$$

See Arntz, Y., Seelig, J. D., Lang, H. P., Zhang, J., Hunziker, P., Ramseyer, J. P., Meyer, E., Hegner, M., and Gerber, C., Label-free protein assay based on a nanomechanical cantilever array, Nanotechnology, 2003, 14, 86-90; and Khaled, A.-R. A., and Vafai, K., Analysis of Deflection Enhancement Using Epsilon Assembly Microcantilevers Based Sensors, Sensors, 2011, 11, 9260-9274.

For this case, the effective elastic modulus varies with the elastic modulus according to the following relationship:

$$Y = \frac{E}{1 - v} \qquad Eq . ( 11 )$$

Δσ can be considered to vary along the microcantilever length according to the following relationship:

$$Δ σ = Δ {{σ}_{o} ( \frac{x}{L} )}^{n} \qquad Eq . ( 12 )$$

where n is the model index. This variation is expected as the analyte concentration in the surrounding environment increases as the distance from the microcantilever base increases. As such, the solution of Equation (1) denoted by z_{Δσ}(x) subject to boundary conditions given by Equations (3(a,b)) can then be expressed as:

$${z}_{Δ σ} ( x ) = 6 ( \frac{1}{{n}^{2} + 3 n + 2} ) \times ( \frac{1 - v}{E} ) Δ {{σ}_{o} ( \frac{L}{d} )}^{2} {( \frac{x}{L} )}^{n + 2} \qquad Eq . ( 13 )$$

The maximum deflection due to analyte adhesion is obtained from Equation (13) by substituting x=L. It is equal to:

$${z}_{Δ σ ma x} = 6 ( \frac{1}{{n}^{2} + 3 n + 2} ) \times ( \frac{1 - v}{E} ) Δ {{σ}_{o} ( \frac{L}{d} )}^{2} \qquad Eq . ( 14 )$$

Equation (14) is reducible to Stoney's equation when n is set to be equal to n=0.

Define the prescribed surface stress deflection indicator Z_{Δσ} as the ratio of the maximum microsensor deflection per maximum differential surface stress under the given prescribed surface stresses. Using Equations (12) and (14), Z_{Δσ} can be shown to be equal to:

$${Z}_{Δ σ} ≡ \frac{{( {z}_{Δ σ} )}_{m ax }}{Δ {σ}_{o}} = 6 ( \frac{1}{{n}^{2} + 3 n + 2} ) \times ( \frac{1 - v}{E} ) {( \frac{L}{d} )}^{2} \qquad Eq . ( 15 )$$

### 1.1.2. The Disturbance in the Deflections of the Typical Rectangular Microcantilever

The one degree of freedom model that can best describe the disturbance in the tip deflection of the typical rectangular microcantilever, z_{d}, is shown in the following differential equation:

$${m}_{eff , 1} \frac{{d}^{2} {z}_{d}}{d {t}^{2}} + {k}_{eff , 1} {z}_{d} = {P}_{o} {ω}^{2} \times cos ( ω t ) \qquad Eq . ( 16 )$$

See Khaled, A.-R. A., Vafai, K., Yang, M., Zhang, X., and Ozkan, C. S., Analysis, control and augmentation of microcantilever deflections in bio-sensing systems, Sens. Actuat. B, 2003, 94, 103-115; Rao S. S., Mechanical Vibrations (5th Edition), Prentice Hall, 2010. USA; and Sader, J., Frequency response of cantilever beams immersed in viscous fluids with applications to the atomic force microscope, Journal of Applied Physics, 1998, 84, 64-76; where m_{eff,1} is the effective mass of the microcantilever at its tip and k_{eff,1} is the effective stiffness of the microcantilever at its tip. ω is the frequency of the dynamic disturbance force and t is time variable. Pₒ is the effective amplitude of the dynamic disturbance force at the tip per square of the frequency of dynamic disturbance. Equation (16) is based on the assumption that the microcantilever is excited in the first mode of vibration and that excitations occur without total energy dissipation. m_{eff1} and k_{eff,1} are given by the following:

$$\begin{matrix}{m}_{eff , 1} = \frac{33}{140} ρ WdL & Eq . ( 17 ) \\ {k}_{eff , 1} = ( \frac{1}{4} ) {EW ( \frac{d}{L} )}^{3} & Eq . ( 18 )\end{matrix}$$

See Rao S. S., Mechanical Vibrations (5th Edition), Prentice Hall, 2010, USA; where p is the density of the microcantilever. The particular solution of the differential equation given by Equation (16) is the following:

$${z}_{d} = \{ \frac{{( ω / {ω}_{o} )}^{2}}{1 - {( ω / {ω}_{o} )}^{2}} \} ( \frac{140}{33} ) ( \frac{{P}_{o}}{ρ WdL} ) \times cos ( ω t ) \qquad Eq . ( 19 )$$

where ωₒ is the first mode natural frequency which is equal to:

$${ω}_{o} = 1.0299 ( \frac{d}{{L}^{2}} ) \sqrt{\frac{E}{ρ}} \qquad Eq . ( 20 )$$

### See Rao S. S., Mechanical Vibrations (5th Edition), Prentice Hall, 2010, USA.

The total maximum deflection of the typical rectangular microcantilever, zₜ, which is the sum of the deflection due to loading of the microcantilever plus the disturbance in the deflection, can be mathematically expressed as follows:

[z_{t,F}z_{r,Δσ}]=[(z_{F})ₘₐₓ+z_{da′(}z_{Δσ})ₘₐₓ+z_{do}] Eq. (21)

where z_{do} is given by:

$${z}_{do} = \{ \frac{{( ω / {ω}_{o} )}^{2}}{\vert 1 - {( ω / {ω}_{o} )}^{2} \vert} \} ( \frac{140}{33} ) ( \frac{{P}_{o}}{ρ WdL} ) \qquad Eq . ( 22 )$$

Define the clearness indicator of the microsensor deflection signal (X_{D}) as the ratio of maximum deflection due to loading type D, where D can be F or Δσ loading types to the sum of that deflection plus the amplitude of the maximum disturbance in the deflection. As such, X_{F} and X_{Δσ} can be shown to be equal to:

$$\begin{matrix}{χ}_{F} = \frac{\vert 1 - {( ω / {ω}_{o} )}^{2} \vert}{\vert 1 - {( ω / {ω}_{o} )}^{2} \vert + {( ω / {ω}_{o} )}^{2} ( \frac{{P}_{o} {ω}_{o}^{2}}{F} )} & Eq . ( 23 ) \\ {χ}_{Δ σ} = \frac{\vert 1 - {( ω / {ω}_{o} )}^{2} \vert}{\begin{matrix}\vert 1 - {( ω / {ω}_{o} )}^{2} \vert + \\ ( \frac{2}{3} ) ( {n}^{2} + 3 n + 2 ) {( ω / {ω}_{o} )}^{2} \times ( \frac{{P}_{o} {ω}_{o}^{2} ( L / d )}{Δ {σ}_{o} W [ 1 - v ]} )\end{matrix}} & Eq . ( 24 )\end{matrix}$$

1.2. The Rectangular Microcatilever with Long-Slit

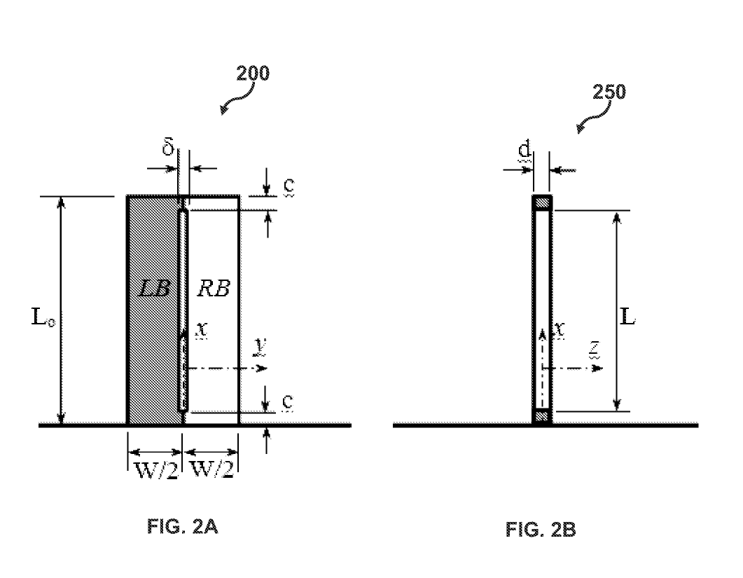

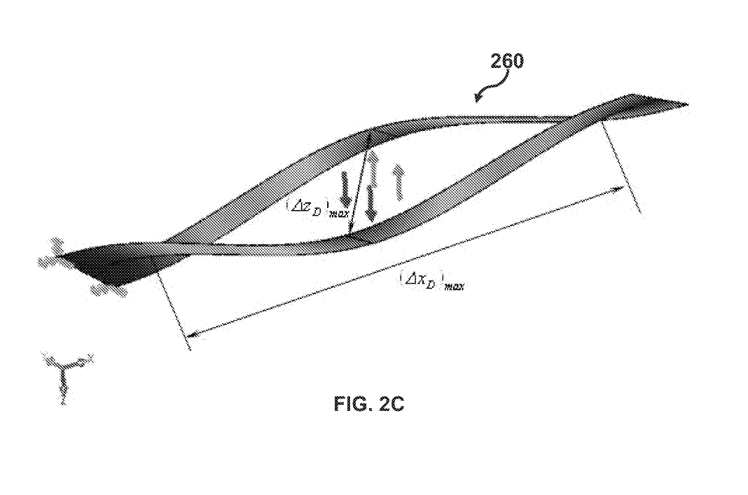

FIGS. 2A and 2B illustrate top and side views 200 and 250 of a typical rectangular microcantilever with long-slit in a corresponding coordinate system respectively, in accordance with the disclosed embodiments. The thickness of the microcantilever and the slit is d. The slit width is while its length is L where δ<<L. The microcantilever length is Lₒ. The side beams on left and right sides of the slit have the same width which is equal to W/2. Each side beam has an area moment of inertia I given by:

$$I = \frac{1}{24} {Wd}^{3} \qquad Eq . ( 25 )$$

1.2.1. Deflections of the Rectangular Microcailever with Long-Slit

FIG. 2C illustrates a deflection profile 260 of rectangular microcantilever with long-slit with major deflection quantities. The length of the microcantilever with long-slit is considered to be much larger than its width. As such, the Hooks law for small deflections can be used to relate the microcantilever deflection at a given cross-section to the effective elastic modulus Y of the microcantilever and the internal bending moment M acting on that section. See Khaled, A.-R. A., and Vafai, K., Analysis of Deflection Enhancement Using Epsilon Assembly Microcantilevers Based Sensors, Sensors, 2011, 11, 9260-9274. It is given by Equation (1).

### Concentrated Force Loadings:

Let the middle cross-section of the beam on the left side of the long-slit be loaded by a normal concentrated force of magnitude F and in the direction of the positive z-axis. On the other hand, the beam on the right side of the long-slit is considered to be loaded by a normal concentrated force of magnitude F, but in the direction of the negative z-axis. When c≦x≦c+L/2, the internal bending moment M distributions on the left side beam (LB) and the right beam (RB) can be shown to be equal to the following:

$$M = ( \frac{1}{8} ) FL \times [ \begin{matrix}1 + 4 ( \frac{c}{L} - \frac{x}{L} ) , & for LB \\ 4 ( \frac{x}{L} - \frac{c}{L} ) - 1 , & for RB ,\end{matrix} \\ c \leq x \leq c + L / 2 \qquad Eq . ( 26 )$$

Accordingly, Equation (1) changes to the following:

$$\frac{{d}^{2} {z}_{F}}{d {x}^{2}} = 3 ( \frac{FL}{{EWd}^{3}} ) \times [ \begin{matrix}1 + 4 ( \frac{c}{L} - \frac{x}{L} ) , & for LB \\ 4 ( \frac{x}{L} - \frac{c}{L} ) - 1 , & for RB ,\end{matrix} \\ c \leq x \leq c + L / 2 \qquad Eq . ( 27 )$$

The boundary conditions of Equation (27) are given by:

$${{Left beam \text{:} {z}_{F} ( x = c ) = \frac{d {z}_{F}}{d x} \vert}_{x = c} = 0 \\ Right beam \text{:} {z}_{F} ( x = c ) = \frac{d {z}_{F}}{d x} \vert}_{x = c} = 0 \qquad Eq . ( 28 a \text{-} d )$$

The magnitude of the maximum stress occurs at (x,z)=(c,±d/2). It is denoted by σ_{oF}. Using Equation (4), σ_{oF} can be shown to be equal to:

$${σ}_{oF} = ( \frac{3}{2} ) \frac{FL}{{Wd}^{2}} \qquad Eq . ( 29 )$$

The solution of Equation (1), denoted by z_{F}(x), can be expressed as:

$${z}_{F} = 3 ( \frac{{FL}^{3}}{{EWd}^{3}} ) \times [ \begin{matrix}\begin{matrix}( \frac{2}{3} ) \{ {\overline{c}}^{3} - {\overline{x}}^{3} \} + ( \frac{1}{2} ) \{ {\overline{c}}^{2} + {\overline{x}}^{2} \} - \\ \overline{cx} \{ 1 + 2 \overline{c} - 2 \overline{x} \} ,\end{matrix} & for LB \\ \begin{matrix}( \frac{2}{3} ) \{ {\overline{x}}^{3} - {\overline{c}}^{3} \} - ( \frac{1}{2} ) \{ {\overline{c}}^{2} + {\overline{x}}^{2} \} + \\ \overline{cx} \{ 1 + 2 \overline{c} - 2 \overline{x} \} ,\end{matrix} & for LB\end{matrix} \qquad Eq . ( 30 )$$

where x̅=x/L and c̅=c/L. The maximum deflections of LB and SB which occurs at x=c+L/2 can then be found. They are equal to the following:

$${( {z}_{F} )}_{ma x} = ( \frac{1}{8} ) ( \frac{{FL}^{3}}{{EWd}^{3}} ) \times [ \begin{matrix}1 , & for LB \\ - 1 , & for RB\end{matrix} \qquad Eq . ( 31 )$$

If the position of RB above the concentrated load is taken as the datum of the rectangular microcantilever with long-slit, then the maximum deflection in that microcantilever denoted by (Δz_{F})ₘₐₓ as shown in FIG. 2C will be:

$${( Δ {z}_{F} )}_{ma x} = {( {z}_{F} )}_{ma x} {{\vert}_{LB} - {( {z}_{F} )}_{ma x} \vert}_{RB} = ( \frac{1}{4} ) ( \frac{F}{EW} ) {( \frac{L}{d} )}^{3} \qquad Eq . ( 32 )$$

The deflection of LB and RB in the positive z-direction and negative z-direction, respectively, causes an opening in the long-slit along the x-z plane. This opening has its maximum width along the z-axis equals to (Δz_{F})ₘₐₓ. The opening maximum length along the x-axis denoted by (Δx_{F})ₘₐₓ as shown in FIG. 2C can be obtained by the following equation:

(Δx_{F})ₘₐₓ={L−2(x|_{z}_{_{F}}_{=d/2}−c)}_{LB}={L=2(x|ₓ_{_{F}}_{=-d/2}−c)}_{LB} Eq(33)

By using Equation (30) and the solution of the cubic equation, (Δx_{F})ₘₐₓ, can be shown to be equal to the following:

$${\frac{{( Δ {x}_{F} )}_{ma x}}{L} = cos ( \frac{1}{3} [ 2 π - {cos}^{- 1} ( - 1 + \frac{d}{{{( {z}_{F} )}_{ma x} \vert}_{LB}} ) ] ) + \frac{1}{2} , \\ {( {z}_{F} )}_{ma x} \vert}_{LB} \geq \frac{d}{2} \qquad Eq . ( 34 )$$

As (Δx_{F})ₘₐₓ>(Δz_{F})ₘₐₓ when (Δz_{F})ₘₐₓ|_{LB}>>d/2, the concentrated force deflection indicator Z_{F} of the microcantilever with long-slit can be redefined as the ratio of (Δx_{F})ₘₐₓ per maximum stress. It is equal to:

$${Z}_{F} ≡ \frac{{( Δ {x}_{F} )}_{ma x}}{{σ}_{oF}} = \frac{L}{{{( {z}_{F} )}_{ma x} \vert}_{LB}} ( \frac{1}{24} ) ( \frac{{L}^{2}}{Ed} ) \{ 1 + 2 \times cos ( \frac{1}{3} [ 2 π - {cos}^{- 1} ( - 1 + \frac{d}{{{( {z}_{F} )}_{ma x} \vert}_{LB}} ) ] ) \qquad Eq . ( 35 )$$

Define the first detection enhancement indicator of the rectangular microcantilever with log-slit due to concentrated force loading γ_{1,F} as the ratio of Z_{F} indicator of the rectangular microcantilever with log-slit to the Z_{F} indicator of the typical rectangular microcantilever. As such, γ_{1,F} is equal to:

$${γ}_{1 , F} = \{ \frac{d}{{{( {z}_{F} )}_{ma x} \vert}_{LB}} \} ( \frac{L}{d} ) ( \frac{1}{16} ) \{ 1 + 2 \times cos ( \frac{1}{3} [ 2 π - {cos}^{- 1} ( - 1 + \frac{d}{{{( {z}_{F} )}_{m ax} \vert}_{LB}} ) ] ) \} \qquad Eq . ( 36 )$$

The following Table 2 provides the maximum value of rectangular microcantilever with long-slit side beams deflection that produces detection enhancement indicator due to concentrated force loading larger than unity.

TABLE 2
(d/L)

| 10⁻² | 10⁻³ | 10⁻⁴ | 10⁻⁵ |
|---|---|---|---|
| 10.882 | 120.29 | 1235.4 | 12454 |

[(z_{F})ₘₐₓ|_{LB}/d]_{γ}_{_{1,F}}₌₁ = 0.10243 × (d/L)^{-1.01874}

### Prescribed Differential Surface Stress:

When one surface of LB is coated with a thin film of receptor, it will bend if analyte molecules adhere on that layer. This adhesion causes a difference in the surface stresses across the microcantilever section (Δσ). On the other hand, RB will bend in the opposite direction if the receptor coating is placed on the surface opposite to that of the LB coated surface. The relation between the magnitude of the internal bending moment M at each cross-section of LB and RB and Δσ is given by Equation (14). Let Δσ be considered to vary along the microcantilever length according to the following relationship:

$$Δσ = Δ {{σ}_{o} ( \frac{x}{L} - \frac{c}{L} )}^{n} \qquad Eq . ( 37 )$$

See Arntz, Y., Seelig, J. D., Lang, H. P., Zhang, J., Hunziker, P., Ramseyer, J. P., Meyer, E., Hegner, M., and Gerber, C., Label-free protein assay based on a nanomechanical cantilever array, Nanotechnology, 2003, 14, 86-90; and Khaled, A.-R. A., and Vafai, K., Analysis of Deflection Enhancement Using Epsilon Assembly Microcantilevers Based Sensors, Sensors, 2011, 11, 9260-9274.

The effective elastic modulus for this case is shown in Equation (15). Accordingly, Equation (1) changes to the following:

$$\frac{{d}^{2} {z}_{Δ σ}}{d {x}^{2}} = 6 ( \frac{1 - v}{{Ed}^{2}} ) Δ {{σ}_{o} ( \frac{x}{L} - \frac{c}{L} )}^{n} \times [ \begin{matrix}1 , & for LB \\ - 1 , & for RB\end{matrix} \qquad Eq . ( 38 )$$

The boundary conditions of Equation (38) are given by:

$${{Left beam \text{:} {z}_{Δ σ} ( x = c ) = \frac{d {z}_{Δ σ}}{d x} \vert}_{x = c} = 0 \\ Right beam \text{:} {z}_{Δ σ} ( x = c ) = \frac{d {z}_{Δ σ}}{d x} \vert}_{x = c} = 0 \qquad Eq . ( 39 a \text{-} d )$$

The solution of Equation (1) can be expressed as:

$${z}_{Δ σ} ( x ) = 6 ( \frac{1}{{n}^{2} + 3 n + 2} ) \times ( \frac{1 - v}{E} ) Δ {{σ}_{o} ( \frac{L}{d} )}^{2} {( \overline{x} - \overline{c} )}^{n + 2} \times [ \begin{matrix}1 , & for LB \\ - 1 , & for RB\end{matrix} \qquad Eq . ( 40 )$$

where x̅=x/L and c̅=c/L. If the position of the midsection of RB is taken as the datum of the rectangular microcantilever with long-slit, then the maximum deflection in that microcantilever denoted by (Δz_{Δσ})ₘₐₓ will be:

$${( Δ {z}_{Δ σ} )}_{max} = {( {z}_{Δ σ} )}_{max} {\vert}_{LB} - {( {z}_{Δ σ} )}_{max} {\vert}_{RB} = 3 {( \frac{1}{2} )}^{n} ( \frac{1}{{n}^{2} + 3 n + 2} ) \times ( \frac{1 - v}{E} ) Δ {{σ}_{o} ( \frac{L}{d} )}^{2} \qquad Eq . ( 41 )$$

The deflection of LB and RB in the positive z-direction and negative z-direction, respectively, causes an opening in the long-slit along the x-z plane. This opening has its maximum width along the z-axis equals to (Δz_{F})ₘₐₓ. The opening maximum length along the x-axis denoted by (Δx_{F})ₘₐₓ can be obtained by the following equation:

(Δx_{Δσ})ₘₐₓ={L−2(x|_{z}_{_{Δσ}}_{=d/2}−c)}↑_{LB}={L−2(x|_{z}_{_{Δσ}}_{=d/2}−c)}|_{LB} Eq. (42)

By using Equation (40) and the solution of the quadratic equation, (Δx_{Δσ})ₘₐₓ can be shown to be equal to the following:

$$\frac{{( Δ {x}_{Δ σ} )}_{max}}{L} = 1 - 2 {\{ \frac{( {n}^{2} + 3 n + 2 )}{12} \times [ \frac{Ed / Δ {σ}_{o}}{( 1 - v ) {( L / d )}^{2}} ] \}}^{\frac{1}{n + 2}} \qquad Eq . ( 43 )$$

As (Δx_{Δσ})ₘₐₓ>(Δz_{Δσ})ₘₐₓ when (Δx_{Δσ})ₘₐₓ|_{LB}>>d2, the concentrated force deflection indicator Z_{Δσ} of the microcantilever with long-slit can be redefined as the ratio of (Δx_{Δσ})ₘₐₓ per maximum stress. It is equal to:

$${Z}_{Δ σ} = \frac{{( Δ {x}_{Δ σ} )}_{max}}{Δ {σ}_{o}} = \frac{1 - {( \frac{d}{2 {( {z}_{Δ σ} )}_{max} {\vert}_{LB}} )}^{\frac{1}{n + 2}}}{Δ {σ}_{o} / L} \qquad Eq . ( 44 )$$

Define the first detection enhancement indicator of the rectangular microcantilever with long-slit due to prescribed differential surface stress loading γ_{Δσ} as the ratio of Z_{Δσ} indicator of the rectangular microcantilever with log-slit to the Z_{Δσ} indicator of the typical rectangular microcantilever. As such, γ_{Δσ} is equal to:

$${γ}_{Δ σ} = {( \frac{1}{2} )}^{n + 1} ( \frac{L}{d} ) ( \frac{d}{2 {( {z}_{Δ σ} )}_{max} {\vert}_{LB}} ) \{ 1 - {( \frac{d}{2 {( {z}_{Δ σ} )}_{max} {\vert}_{LB}} )}^{\frac{1}{n + 2}} \} \qquad Eq . ( 45 )$$

The following Table 3 provides the maximum value of rectangular microcantilever with long-slit side beams deflection that produces detection enhancement indicator due to prescribed differential surface stress loading larger than unity.

ABLE 3

| n | (d/L) | [(z_{ΔG})ₘₐₓ\|_{LB}/d]_{γ}_{_{ΔG}}₌₁ |
|---|---|---|
| n = 0 | 10⁻² | 21.157 |
| | 10⁻³ | 238.55 |
| | 10⁻⁴ | 2464.4 |
| | 10⁻⁵ | 24888 |
| n = 0.5 | 10⁻² | 12.854 |
| | 10⁻³ | 159.14 |
| | 10⁻⁴ | 1699.3 |
| | 10⁻⁵ | 17408 |
| n = 1.0 | 10⁻² | 7.4114 |
| | 10⁻³ | 103.90 |
| | 10⁻⁴ | 1155.5 |
| | 10⁻⁵ | 12067 |
| n = 1.5 | 10⁻² | 3.9373 |
| | 10⁻³ | 66.535 |
| | 10⁻⁴ | 775.55 |
| | 10⁻⁵ | 8288.2 |

1.2.2. The Disturbance in the Deflections of the Rectangular Microcantilever with Long-Slit

The one degree of freedom model that can best describe the disturbance in the deflections at the midsections of LB and EB of the rectangular microcantilever of the long-slit, z_{d}, is shown in the following differential equation:

$${m}_{eff , 2} \frac{{d}^{2} {z}_{d}}{d {t}^{2}} + {k}_{eff , 2} {z}_{d} = {P}_{o} {ω}^{2} [ \begin{matrix}cos ( ω t ) , for LB \\ cos [ π ( W / λ ) - ω t ] , for RB\end{matrix} \qquad Eq . ( 46 )$$

See Khaled, A.-R. A., Vafai, K., Yang, M., Zhang, X., and Ozkan, C. S., Analysis, control and augmentation of microcantilever deflections in bio-sensing systems, Sens. Actuat. B, 2003, 94, 103-115; Rao S. S., Mechanical Vibrations (5th Edition), Prentice Hall, 2010, USA; and Sader, J., Frequency response of cantilever beams immersed in viscous fluids with applications to the atomic force microscope, Journal of Applied Physics, 1998, 84, 64-76: where m_{eff,2} is the effective mass of the LB or RB at their midsections, k_{eff,2} is the effective stiffness of the LB or RB at their midsections, Pₒ is the effective amplitude of the dynamic disturbance force at LB or RB midsections per square of disturbance frequency, and ω is the frequency of the dynamic disturbance force. The variable t and quantity λ are the time variable and the wave length of the dynamic disturbance, respectively. Equation (46) is based on the assumption that the microcantilever is excited in the first mode of vibration without total energy dissipation. m_{eff,2} and k_{eff,2} can be shown to be equal to the following:

$$\begin{matrix}{m}_{eff , 2} = 0.19179 ρ WdL & Eq . ( 47 ) \\ {k}_{eff , 2} = 8 {EW ( \frac{d}{L} )}^{3} & Eq . ( 48 )\end{matrix}$$

See Rao S. S., Mechanical Vibrations (5th Edition), Prentice Hall, 2010, USA.

The particular solution of the differential equation given by Equation (46) is the following:

$${z}_{d} = 5.2140 \{ \frac{{( ω / {ω}_{s} )}^{2}}{1 - {( ω / {ω}_{s} )}^{2}} \} ( \frac{{P}_{o}}{ρ W d L} ) \times [ \begin{matrix}cos ( ω t ) , for LB \\ cos [ π ( W / λ ) - ω t ] , for RB\end{matrix} \qquad Eq . ( 49 )$$

where ωₛ is the first mode natural frequency which is equal to:

$${ω}_{s} = 6.4585 ( \frac{d}{{L}^{2}} ) \sqrt{\frac{E}{ρ}} = 6.2710 {ω}_{o} \qquad Eq . ( 50 )$$

Thus, the total maximum deflection of LB and RB denoted by z_{tL} and z_{tR}, respectively, is the sum of the deflection due to force loadings of these beams plus the disturbance in the deflection at time equal to t=0. They can be mathematically expressed as follows:

$$[ \begin{matrix}{z}_{t L , F} \\ {z}_{t L , Δ σ}\end{matrix} ] = [ \begin{matrix}{( {z}_{FL} )}_{max} + {z}_{d 1} \\ {( {z}_{Δ σ R} )}_{max} + {z}_{d 1} cos [ π ( W / λ ) ]\end{matrix} ] \qquad Eq . ( 51 )$$

where z_{d1} is given by:

$${z}_{d 1} = 5.2140 \{ \frac{{( ω / {ω}_{s} )}^{2}}{\vert 1 - {( ω / {ω}_{s} )}^{2} \vert} \} ( \frac{{P}_{o}}{ρ W d L} ) \qquad Eq . ( 52 )$$

Since the position of RB at its midsection is taken as the maximum total deflection in that microcantilever denoted by (Δz_{t,F})ₘₐₓ or (Δz_{tΔσ})ₘₐₓ will be:

$$[ \begin{matrix}{( Δ {z}_{t , f} )}_{max} \\ {( Δ {z}_{t , Δ σ} )}_{max}\end{matrix} ] = [ \begin{matrix}{( {z}_{t , F} )}_{max} {\vert}_{LB} - {( {z}_{t , F} )}_{max} {\vert}_{RB} \\ {( {z}_{t , Δ σ} )}_{max} {\vert}_{LB} - {( {z}_{t , Δ σ} )}_{max} {\vert}_{RB}\end{matrix} ] = [ \begin{matrix}{( Δ {z}_{F} )}_{max} \\ {( Δ {z}_{Δ σ} )}_{max}\end{matrix} ] + {z}_{d 1} ( 1 - cos [ π ( W / λ ) ] ) \times [ \begin{matrix}1 \\ 1\end{matrix} ] \qquad Eq . ( 53 )$$

By the inspection of Equations (21) and (53), the maximum ratio of the amplitude of the disturbance in the deflection of the rectangular microcantilever with long-slit to that of the typical microcantilever is lower than 0.01 when the wave length satisfys the following constraints:

$$\frac{1}{2 m + 0.28713} < ( \frac{λ}{W} ) < \frac{1}{2 m - 0.28713} , m = 1 , 2 , 3 , K ∞ ; \\ ( \frac{λ}{W} ) > 3.4827 \qquad Eq . ( 54 )$$

The clearness indicator of the deflection signal of the present microsensor (X_{D}) is redefined here as the ratio of maximum deflection due to loading type D, where D can be F or Δσ loading types to the sum of that deflection plus the maximum disturbance in the deflection. As such, X_{F} and X_{Δσ} for this case are equal to:

$$\begin{matrix}{χ}_{F} = \frac{\vert 1 - 0.02543 {( ω / {ω}_{o} )}^{2} \vert}{\begin{matrix}\vert 1 - 0.02543 {( ω / {ω}_{o} )}^{2} \vert + \\ 0.5 {( ω / {ω}_{o} )}^{2} ( \frac{{P}_{o} {ω}_{o}^{2}}{F} ) ( 1 - cos [ π ( W / λ ) ] )\end{matrix}} & Eq . ( 55 ) \\ {χ}_{Δ σ} = \frac{\vert 1 - 0.02543 {( ω / {ω}_{o} )}^{2} \vert}{\begin{matrix}\vert 1 - 0.02543 {( ω / {ω}_{o} )}^{2} \vert + ( \frac{1}{3} ) ( {2}^{n - 3} ) ( {n}^{2} + 3 n + 2 ) \\ {( ω / {ω}_{o} )}^{2} ( \frac{{P}_{o} {ω}_{o}^{2} ( L / d )}{Δ {σ}_{o} W [ 1 - v ]} ) ( 1 - cos [ π ( W / λ ) ] )\end{matrix}} & Eq . ( 56 )\end{matrix}$$

To compare between the clearness indicator of the rectangular microcantilever with long-slit and that of typical microcantilever, the second detection enhancement indicator (γ_{2,D}) is defined as the ratio of X_{D}- value of the rectangular microcantilever with long-slit to that of the typical rectangular microcantilever where can be either F or Δσ. Mathematically, they are equal to the following:

$$\begin{matrix}{γ}_{2 , F } = \frac{\vert 1 - 0.02543 {( ω / {ω}_{o} )}^{2} \vert \{ \vert 1 - {( ω / {ω}_{o} )}^{2} \vert + {( ω / {ω}_{o} )}^{2} {C}_{1} \}}{\begin{matrix}\vert 1 - {( ω / {ω}_{o} )}^{2} \vert \\ \{ \begin{matrix}\vert 1 - 0.02543 {( ω / {ω}_{o} )}^{2} \vert + 0.5 {( ω / {ω}_{o} )}^{2} {C}_{1} \\ ( 1 - cos [ π ( W / λ ) ] )\end{matrix} \}\end{matrix}} & Eq . ( 57 ) \\ {γ}_{2 , Δ σ} = \frac{\begin{matrix}\vert 1 - 0.02543 {( ω / {ω}_{o} )}^{2} \vert \\ \{ \vert 1 - {( ω / {ω}_{o} )}^{2} \vert + ( \frac{2}{3} ) ( {n}^{2} + 3 n + 2 ) {( ω / {ω}_{o} )}^{2} {C}_{2} \}\end{matrix}}{\begin{matrix}\vert 1 - {( ω / {ω}_{o} )}^{2} \vert \\ \{ \begin{matrix}\vert 1 - 0.02543 {( ω / {ω}_{o} )}^{2} \vert + ( \frac{1}{3} ) ( {2}^{n - 3} ) ( {n}^{2} + 3 n + 2 ) \\ {( ω / {ω}_{o} )}^{2} {C}_{2} ( 1 - cos [ π ( W / λ ) ] )\end{matrix} \}\end{matrix}} & Eq . ( 58 )\end{matrix}$$

where C₁=P₀ω₀²/F and C₂=P₀ω₀²(L/d)/[Δσ₀W(1−v)].

### 2. Results and Discussion

### 2.1. Validation of the Results

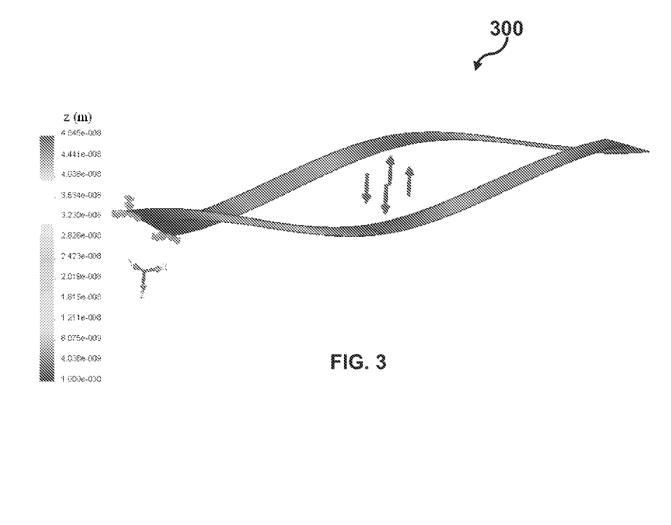

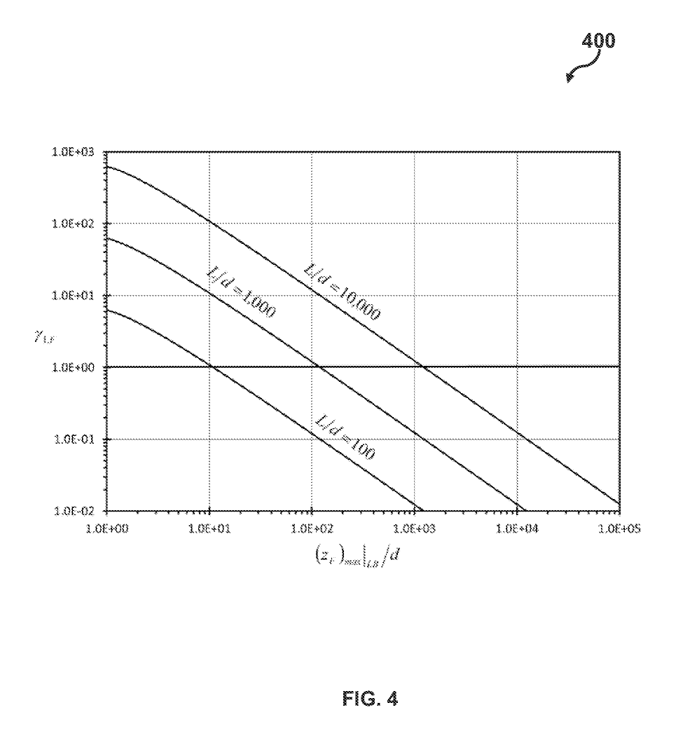

The present analytical methods for the rectangular microcantilever with long-slit were tested against an accurate numerical solution using finite element methods and accounting for all mechanical constraints induced by the geometry. Among these constraints is the torsion effect of the concentrated force and restraining the wrapping of the side beams due to end portions of the microcantilever. The deflection profile 300 for rectangular microcantilever with long-slit with Lₒ=425 μm, L=415 μm, W=60 μm, d=0.4 μm, c=5 μm, and δ=2 μm under concentrated force loading with F=2×10⁻⁹ N is shown in FIG. 3. The microcantilever material was taken to be silicon with E=0.1124N·μm⁻² and a poisons ratio of v=0.28. The maximum deflection of LB is equal to (z_{F})ₘₐₓ=41.4 nm using Equation (31). As can be seen from FIG. 2C, the average deflection of the mid-section of the LB is about (z_{F})ₘₐₓ=46.4 nm. Notice that the maximum error between the numerical and the derived analytical solutions is less than 11 percent. The previous small percentage difference gives more confidence on the obtained results. The generated results of various defined detection performance indicators are presented graphically in FIGS. 4-11.

### 2.2. Discussion of the Results

### 2.2.1. Discussion of the Results of First Detection Enhancement Indicator

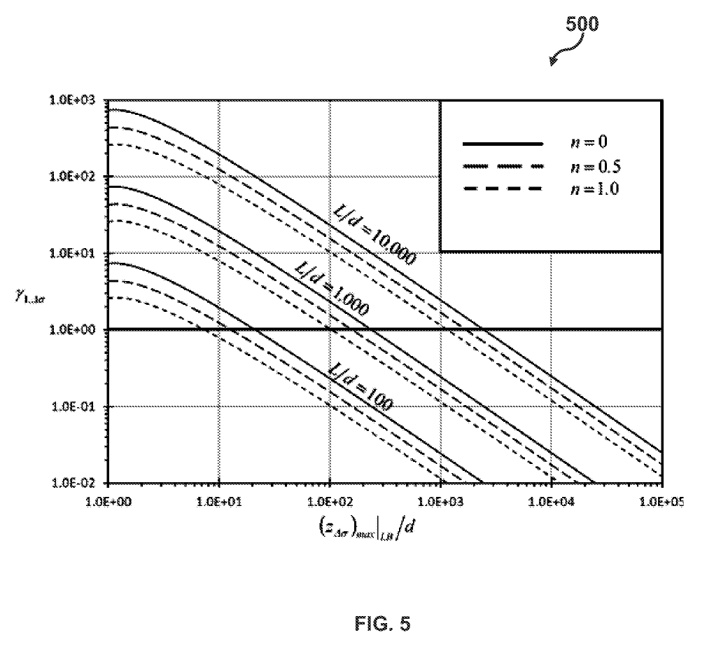

FIGS. 4-5 illustrate graphs 400 and 500 showing the variation of the first detection enhancement indicator of the rectangular microcantilever with long-slit due to concentrated force loading and prescribed surface stress loading (γ_{1,F}, γ_{1,Δσ}), respectively, with maximum side beam relative deflection {(z_{F})ₘₐₓ|_{LB}/d} for different slit profile dimensionless length (L/d). It is noticed that both γ_{1,F} and γ_{1,Δσ} increase as (z_{F})ₘₐₓ|_{LB}/d decreases and as L/d increases. This indicates the superiority of the rectangular microcantilever with long-slit over typical rectangular microcantilever in detection of low analyte concentration especially when long slits are considered. Both figures show that the detection capability of the rectangular microcantilever with long-slit can be more than 100 times that of the typical rectangular microcantilever. Moreover, it is noticed from FIG. 4 that γ_{1,Δσ} is enhanced as n decreases and its maximum value occur when n=0. This means that well-mixed analyte solutions produce better detection capability than weakly mixed ones as n index approaches n=0 when the mixing level increases.

### 2.2.2. Discussion of the Results of Clearness Indicator of Typical Rectangular Microcantilever

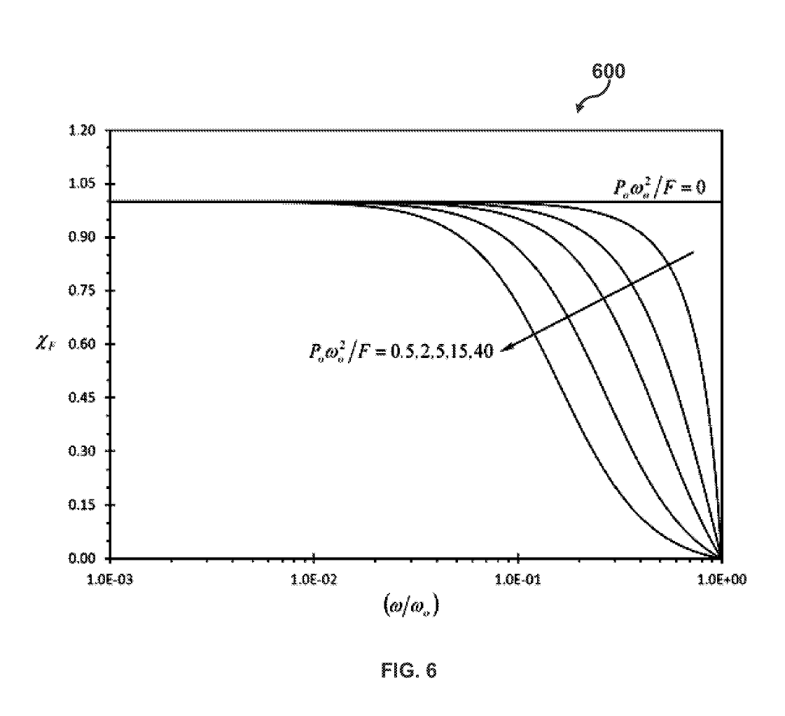

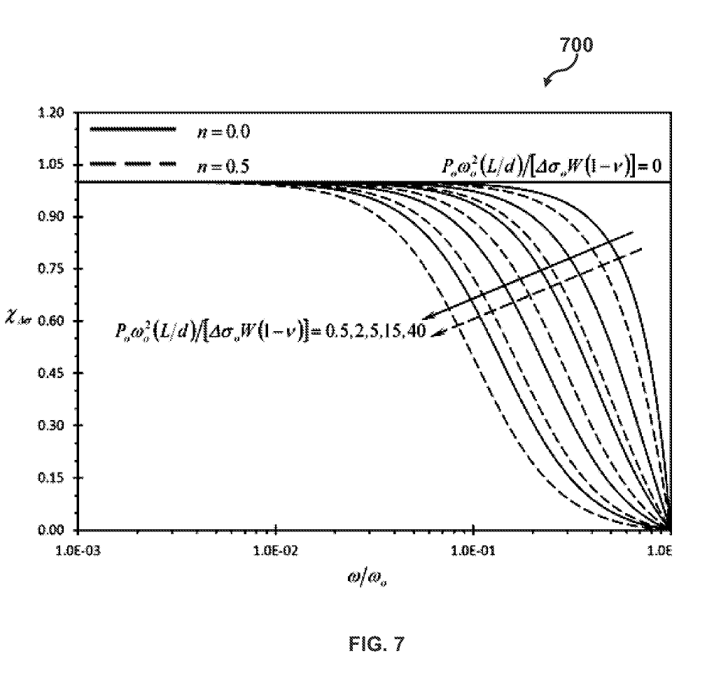

FIGS. 6-7 illustrate graphs 600 and 700 showing the effect of the dimensionless frequency of dynamic disturbance (ω/ωₒ) on the dearness indicator of the deflection signal of the typical rectangular microcantilever due to concentrated force loading and prescribed surface stress loading (X_{F}, X_{Δσ}), respectively. It is noticed that both X_{F} and X_{Δσ} goes to zero as the frequency of dynamic disturbance approaches the fundamental natural frequency (ω/ωₒ=1.0). This indicates that the detection of the typical microcantilever becomes unrecognizable when ω approaches ω=ωₒ. Moreover, both indicators are expected to decrease as the amplitude of dynamic disturbance excitation force per square of disturbance frequency (Pₒ) increases. This behavior is noticeable in FIGS. 6-7. Furthermore, it is seen in FIG. 6 that X_{Δσ} is enhanced as n decreases and its maximum values occur when n=0. Again, this confirms that well-mixed analyte solutions produce lower disturbance levels in the detection signal than weakly mixed ones as n approaches n=0 when the mixing level increases.

### 2.2.3. Discussion of the Results of Second Detection Enhancement Indicator

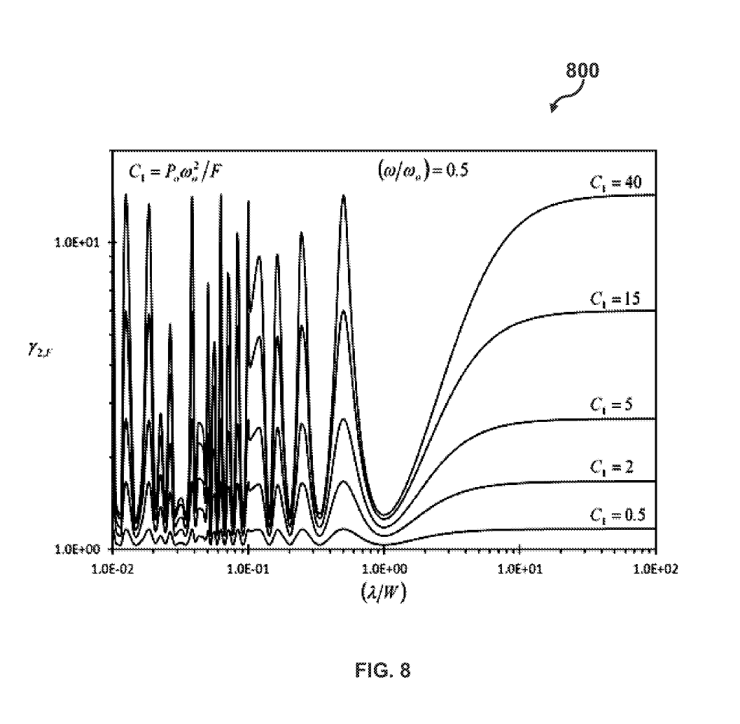

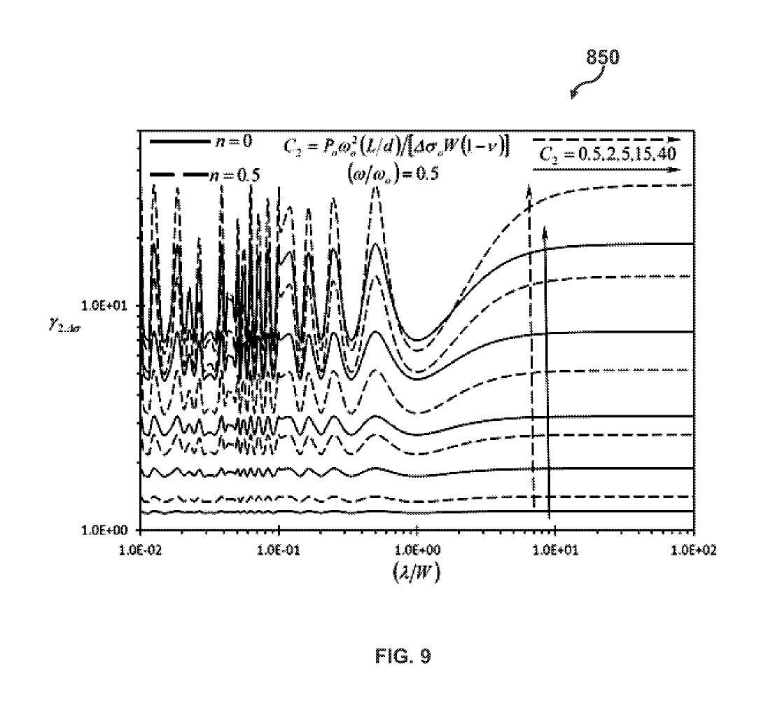

FIGS. 8-9 illustrate graphs 800 and 850 showing the effect of the dimensionless dynamic disturbance wave length (λ/W) on the second detection enhancement indicator of the rectangular microcantilever with long-slit under concentrated force loading and prescribed surface stress loading (γ_{2,F}, γ_{2,Δσ}), respectively. The results of this figure are generated with dimensionless frequency of dynamic disturbance equal to (ω/ωₒ)=0.5. It is noticed that both indicators are always larger than one regardless of the wave length. As such, the detection of the rectangular microcantilever with long-slit is better isolated against dynamic disturbances than the typical rectangular microcantilever. When λ/W≧3.4827, γ_{2,F}, and γ_{2,Δσ} are always increasing as the wave length increases. For small wave length dynamic disturbance, there is a chance that the wave disturbances at the center of LB and RB be of the same phase shift, phase shift difference of π or of phase shift difference between 0 and π. In case of the same phase shift, the disturbance in the detection of the rectangular microcantilever with long-slit is eliminated when subtracting the deflection of RB from that of LB. For the case when π is the phase shift difference, the subtraction process leads to agglomeration in the disturbance in the detection signal as dictated from Equations (57) and (58). When the phase shift difference is between 0 and π, the disturbance in the detection signal becomes more significant as the phase shift difference approaches π. FIGS. 8-9 demonstrate that larger dynamic disturbance forces obtained by larger Pₒ values make the rectangular microcantilever with long-slit more superior than the typical rectangular microcantilever since γ_{2,F} and γ_{2,Δσ} increases as Pₒ increases. Finally, FIG. 9 shows that γ_{2,Δσ} increases as n increases. This is expected because as n increases, the effective force producing the deflection decreases causing a similar effect as that of increasing the C₂ value.

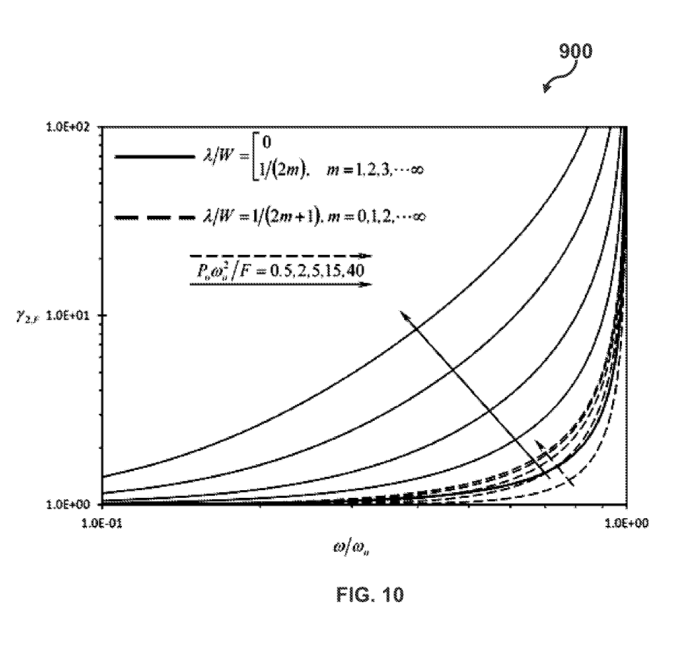

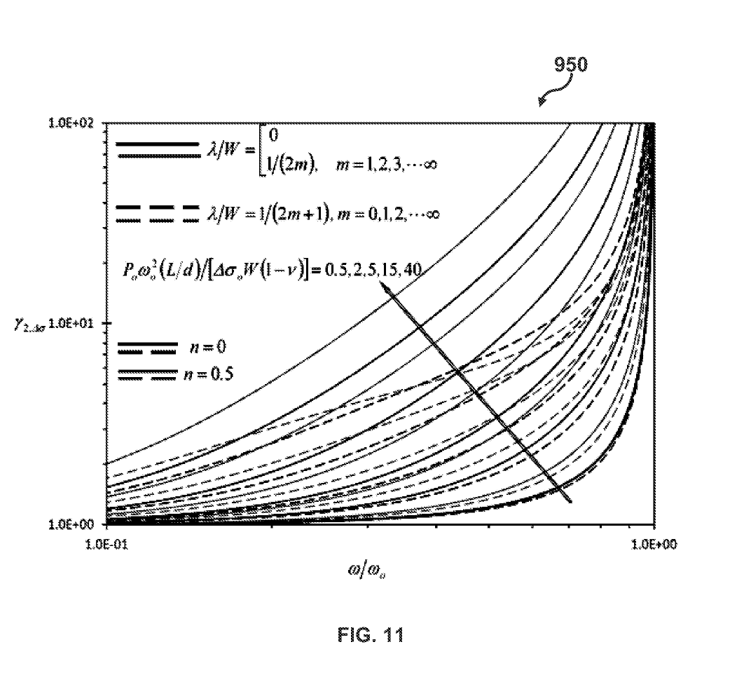

FIG. 10 illustrate graph 900 showing the effect of the dimensionless frequency of dynamic disturbance (ω/ωₒ) on the second detection enhancement indicator of the rectangular microcantilever with long-slit due to concentrated force loading (γ_{2,F}) for two different sets of wave lengths. The set shown for solid lines produce same phase shift for the wave disturbances at the center of LB and RB. For this set, the clearance indicator of the rectangular microcantilever with long-slit is equal to one. That is, the detection quantities are unaffected by dynamic disturbances for this set of wave lengths. As such, γ_{2,F} values are maxima for that set of wave lengths. On the other hand, the set of wave lengths shown for dashed lines produce phase shift difference between the wave disturbances at the center of LB and RB equal to π. For this set, the rectangular microcantilever with long-slit will have the minima values of clearance indicators thus, γ_{2,F} values are minima for the dashed lines. According to FIG. 10, γ_{2,F} is always larger than one regardless of the frequency of dynamic disturbance as long as ω<ωₒ. When ω>ωₒ, the one degree of freedom model cannot be used to determine the disturbance in the deflection and more advanced models are required such as the Euler-Bernoulli beam theory. See Khaled, A.-R. A., and Vafai, K., Analysis of Deflection Enhancement Using Epsilon Assembly Microcantilevers Based Sensors, Sensors, 2011, 11, 9260-9274. The applications of these advanced models on the rectangular microcantilever with long-slit have many complications due to complexity of the geometry. Moreover, γ_{2,F} is always increasing as ω increases. Similar trends are shown in FIG. 11 graph 950, where the rectangular microcantilever with long-slit is under the prescribed surface stress loading. Furthermore, the superiority of the rectangular microcantilever with long-slit over the typical rectangular microcantilever increases as Pₒ increases is shown in FIGS. 10-11 since both γ_{2,F} and γ_{2,Δσ} increase as Pₒ increases.

### 3. Conclusions

An investigation on verifying the advantage of using rectangular microcantilevers with long-slit in microsensing applications was performed in this work based on analytical solutions. The detection capabilities of these microcantilevers were compared against that of typical rectangular microcantilevers under presence of dynamic disturbances. Concentrated force loadings and prescribed surface stress loadings were considered as the sensing driving forces. The theory of linear elasticity for thin beam deflections was used to obtain the detection quantities. The disturbance in these quantities was obtained using the wave propagation and beam vibration theories. The defection profile of the rectangular microcantilever with long-slit was validated against an accurate numerical solution utilizing finite element method with maximum deviation less than 10 percent.

It was found that the detection of the rectangular microcantilevers with long-slit based on its maximum slit opening length can be more than 100 times the maximum deflection of the typical rectangular microcantilever. Furthermore, the disturbance (noise) in the deflection of the microcantilever with long-slit was found to be always smaller than that of the typical microcantilevers regardless of the wave length, force amplitude, and the frequency of the dynamic disturbance. Moreover, well-mixing the analyte solution was found to produce better detection capability and smaller disturbance in the detection of the microcantilever with long-slit than weakly-mixed ones. Eventually, detections of the microcantilevers with long-slit were found to be practically unaffected by dynamic disturbances as long as wave lengths of these disturbances are larger than 3.5 times the width of the microcantilever. Finally, the present work strongly suggests implementation of microcantilevers with long-slit as microsensors in real analyte environments and out of the laboratory testing.

It will be appreciated that variations of the above disclosed apparatus and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

What is claimed is:

1. A method for analyzing detection enhancement of rectangular microcantilevers with long-slit in microsensing applications, said method comprising:
comparing a deflection profile associated at least one rectangular microcantilever with long-slit with that of typical rectangular microcantilevers under static conditions;
comparing a deflection profile associated at least one rectangular microcantilever with long-slit with that of typical rectangular microcantilevers under a presence of dynamic disturbances;
considering various force-loading conditions with respect to said at least one rectangular microcantilever with long-slit;
calculating a plurality of deflection related quantities with respect to said at least one rectangular microcantilever with long-slit based on a linear elasticity; and
obtaining a disturbance in said quantities based on wave propagation and beam vibration theories to derive data indicative of detection enhancement with respect to said at least one rectangular microcantilever with long-slit,

2. The method of claim 1 further comprising validating said defection profile with a long-slit configuration against an accurate numerical solution utilizing a finite element with a maximum deviation of less than 10 percent.

3. The method of claim 1 wherein a detection of said at least one rectangular microcantilever having a long-slit is based on a maximum slit opening length of more than 10 times deflections of said typical rectangular microcantilevers.

4. The method of claim 1 wherein said disturbance in detection quantities of said at least one rectangular microcantilever with a long-slit is smaller than said at least one rectangular microcantilever regardless of a wave length, a force amplitude, and a frequency of a dynamic disturbance.

5. The method of claim 1 wherein detection quantities of said at least one rectangular microcantilever with a long-slit are unaffected by dynamic disturbances as long as the wavelengths of said dynamic disturbances are larger than 3.5 times a width of said at least one rectangular microcantilever.
